(19) United States
(12) Patent Application Publication
(10) Pub. No.: US 2018/0140464 A1
(43) Pub. Date: May 24, 2018

(54) DUAL NOISE ATTENUATION (DNA) HEADSET/EARMUFF

(71) Applicant: Joseph Berto, White City, OR (US)

(72) Inventor: Joseph Berto, White City, OR (US)

(21) Appl. No.: 15/689,127

(22) Filed: Aug. 29, 2017

Related U.S. Application Data
(60) Provisional application No. 62/425,304, filed on Nov. 22, 2016.

(51) Int. Cl. A61F 11/12; A61F 11/14; H04R 1/10

(52) U.S. Cl. CPC A61F 11/12; A61F 11/14; A61F 2011/085; H04R 1/1008; H04R 1/1066; H04R 1/1016

(57) ABSTRACT

A dual attenuation ear (DNA) protecting headset/earmuff, having means for externally adjusting the location of an integral inner earplug into the ear canal of a wearer, useful in high noise environments. In a headgear such as an earmuff, there is typically no way to insert an additional attenuation device, such as an earplug, once the earmuff is placed on the ear. The apparatus includes a pair of toggle levers equipped with replaceable earplugs on the ends, which are integral to the headgear and accessible from outside of the earcup. Manual manipulation of the toggle selectively manipulates the end of the earplug into the wearer's ear, providing additional degrees of ambient sound attenuation. The earmuff device, with concurrent dual protection, can be placed or removed from the ear without requiring a two-step process of first inserting the earplug, and then covering it with the earcup or vise-versa.

### CROSS-REFERENCES TO RELATED APPLICATIONS

This application claims priority to U.S. Provisional Application No. 62/425,304 filed on Nov. 22, 2016, the entire content of which is incorporated herein by reference.

### FIELD OF THE INVENTION

This invention relates generally to the field of headgear and more particularly to an improved hearing protection device (HPD), for example, a dual noise attenuation (DNA) headset/earmuff or a dual input quad headphones or speaker.

### BACKGROUND OF THE INVENTION

Personnel working in high ambient noise environments, such as in factories, aircraft operations, wood processing and the like must be protected from possible damage to their ears. They must also generally maintain sound communication with other workers, and it is therefore necessary to allow the easy removal and re-installation of an HPD. Typical devices found in the prior art provide some form of sound attenuation, including earmuffs and earplugs. In especially noisy environments, those above 100 dBA, it is suggested that the two be used in combination, commonly called “dual protection”. However, dual protection has its own liabilities and limitations.

This invention remedies those issues, providing the ability to quickly install and remove a dual protection device as well as optimize each portion of the device to work with each other for maximum noise attenuation.

### SUMMARY OF THE INVENTION

In consideration of the above issues, it would be desirable to have a headset, which includes an earmuff and an earplug in each of the headset assemblies and wherein the earplug can be easily adjusted to fit within a user's ear canal while the earmuff in place over the ear.

Accordingly, it is the general purpose and object of the present invention to provide simple, inexpensive, lightweight, efficient, fast, simultaneous selective means for providing dual protection, namely an earmuff combined with an earplug, and a method for adjusting the earplug inside the wearers ear while the earmuff is covering the ear, by a single quick external manipulation. In accordance with an exemplary embodiment, selective adjustment of each earplug can be generally accomplished by the individual adjustment of a toggle at each earmuff.

A dual attenuation ear protecting headgear is disclosed, the headgear comprising: a pair of earmuff assemblies; a headband assembly for connecting the pair of earmuff assemblies to one another; and an external means for adjusting a location of an integral inner earplug into the ear canal of a wearer in each of the pair of earmuff assemblies.

A dual attenuation ear protecting headgear is disclosed, the headgear comprising: a pair of earmuff assemblies; a headband assembly for connecting the pair of earmuff assemblies to one another; and a toggle assembly for externally adjusting a location of an integral inner earplug into the ear canal of a wearer in each of the pair of earmuff assemblies.

A dual input quad headphone assembly is disclosed, the headphone assembly comprising; a pair of earmuff assemblies, each of the pair of earmuff assemblies having a speaker configured to receive an audio signal; a headband assembly for connecting the pair of earmuff assemblies to one another; and an external means for adjusting a location of an integral inner earbud into the ear canal of a wearer in each of the pair of earmuff assemblies, each earbud receiving an audio signal.

It is to be understood that both the foregoing general description and the following detailed description are exemplary and explanatory and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a side elevation view of a dual noise attenuation (DNA) headset/earmuff with an ear protecting muff and toggle supporting structure with adjustable integral earplugs according to an exemplary embodiment.

FIG. 2 is a cross-sectional view of FIG. 1 with the earmuffs and toggle supporting structure with adjustable integral earplugs shown in front elevation.

FIG. 3 is another cross-sectional view of FIG. 1 with the earmuffs and toggle supporting structure with adjustable integral earplugs shown in rear elevation.

FIG. 4 is an enlarged fragmentary sectional view of the left ear muff 1 and toggle mount with adjustable integral earplugs of FIGS. 2 and 3.

FIG. 5 is an enlarged fragmentary sectional view of the right ear muff and toggle mount with adjustable integral earplugs of FIGS. 2 and 3.

FIG. 6 is an enlarged fragmentary sectional view of the adjustable integral earplug toggle structure of FIGS. 2 and 3.

FIG. 7 is another enlarged fragmentary sectional view of the adjustable integral earplug toggle structure of FIGS. 2 and 3.

FIG. 8 is a view of the dual noise attenuation headset with ear protecting muff and toggle supporting structure with adjustable integral earplugs as shown from the right.

FIG. 9 is a view of the dual noise attenuation headset with ear protecting muff and toggle supporting structure with adjustable integral earplugs as shown from the right with the headset worn in the reverse position.

FIG. 10 is a side elevation view of a dual input quad speaker headset with an ear protecting muff and toggle supporting structure with adjustable integral earplugs according to an exemplary embodiment.

FIG. 11 is an elevation view of the dual input quad speaker headset of FIG. 10 with an ear protecting muff and toggle supporting structure with adjustable integral earplugs according to an exemplary embodiment.

FIG. 12 is an elevation view of a left headphone assembly.

FIG. 13 is a side elevation view of the left headphone assembly as shown in FIG. 12.

FIG. 14 is an enlarged fragmentary sectional view of the left headphone assembly.

FIG. 15 is an enlarged fragmentary sectional view of the right headphone assembly.

FIGS. 16A-16D are enlarged fragmentary sectional views of the adjustable integral earplug toggle structure of FIGS. 11 and 12.

FIG. 17 is a view of the dual input quad speaker headset with ear protecting muff and toggle supporting structure with adjustable integral earplugs as shown from the right.

FIG. 18 is a view of the dual input quad speaker headset with ear protecting muff and toggle supporting structure with adjustable integral earplugs as shown from the right with the headset worn in the reverse position.

### DETAILED DESCRIPTION

Reference will now be made in detail to the present preferred embodiments of the invention, examples of which are illustrated in the accompanying drawings. Wherever possible, the same reference numbers are used in the drawings and the description to refer to the same or like parts.

In accordance with an exemplary embodiment, a dual attenuation ear protecting headgear, having means for adjusting the location of an integral inner earplug into the ear canal of a wearer, useful in high noise environments. In a headgear such as an earmuff, there is typically no way to insert an additional attenuation device, such as an earplug, once the earmuff is placed on the ear. The headset or apparatus can include a pair of toggle levers equipped with replaceable earplugs on the ends, which are integral to the headgear and accessible from outside of the earcup. Manual manipulation of the toggle selectively manipulates the end of the earplug into the wearer's ear, providing additional degrees of ambient sound attenuation. The earmuff device, with concurrent dual protection, can be placed or removed from the ear without requiring a two-step process of first inserting the earplug, and then covering it with the earcup or vise-versa.

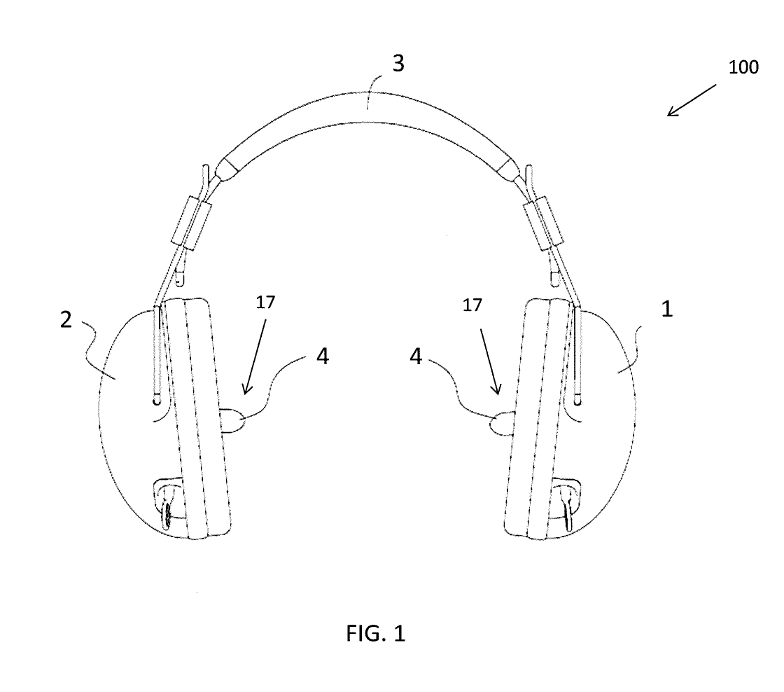

FIG. 1 is a side elevation view of a dual noise attenuation headset 100 with an ear protecting muff and toggle supporting structure with adjustable integral earplugs according to an exemplary embodiment. As shown in FIG. 1, the dual noise attenuation headset 100 includes a left hand earmuff assembly (or first earmuff assembly) 1, a right hand earmuff assembly (or second earmuff assembly) 2, a headband assembly 3, and a toggle assembly 4 for each of the right and left hand earmuff assembly 1, 2. As shown, the headband assembly 3 attaches the right and the left earmuff assemblies 1, 2 to one another. In accordance with an exemplary embodiment, the headband assembly 3 is configured to be worn on the top of the user's head. Each of the toggle assemblies 4 can be configured to allow a user of the dual noise attenuation headset 100 to be able to externally adjust the earplug 17 into the respective ear canal of the user during use.

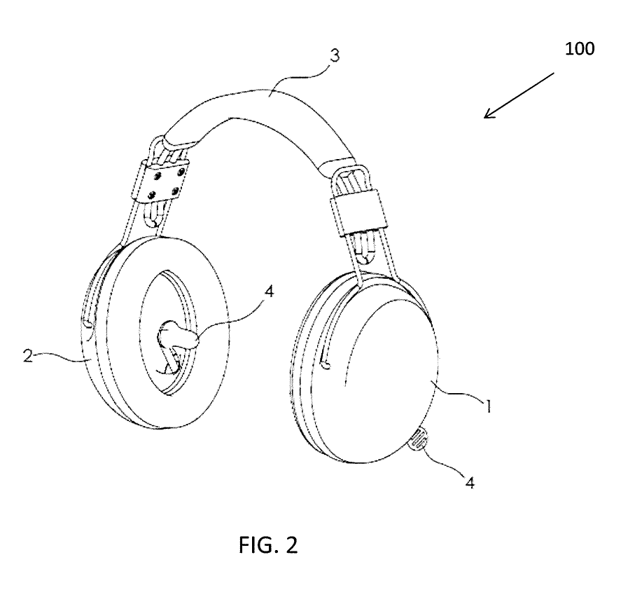

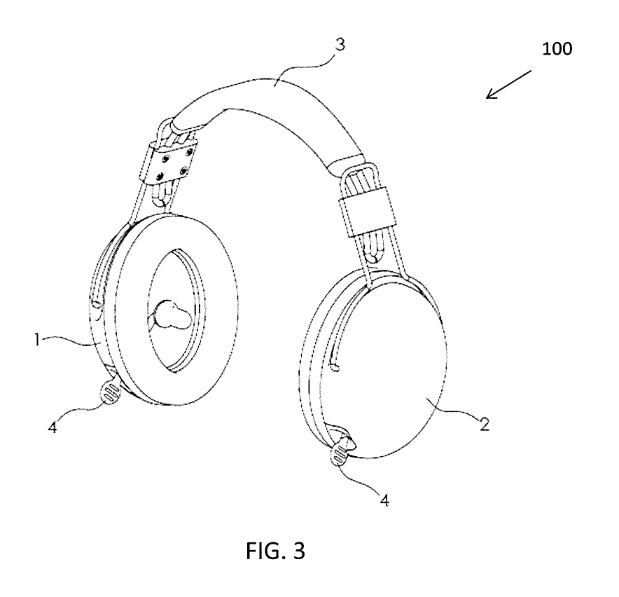

FIGS. 2 and 3 are cross-sectional views of FIG. 1 with the earmuffs and toggle supporting structure 4 with adjustable integral earplugs 17 shown in front and rear elevation respectively. As shown in FIGS. 2 and 3, the toggle assembly 4 is configured to fit within each of the right and left hand earmuff assembly 1, 2, and extends outward to allow the user to adjust the earplug 17.

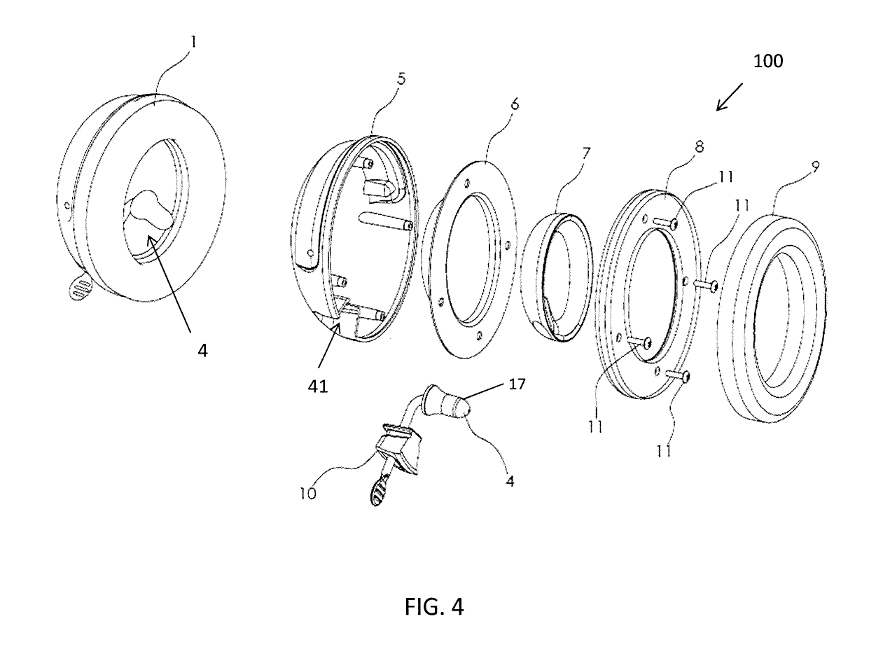

FIG. 4 is an enlarged fragmentary sectional view of the left ear muff 1 and toggle mount 4 with adjustable integral earplugs 17 of FIGS. 2 and 3. As shown in FIG. 4, the left earmuff assembly 1 can include a left hand ear cup assembly 5, a left hand attenuation foam mount 6, a left hand attenuation foam 7, an earmuff cushion mount 8, an earmuff cushion 9, a left hand toggle mount 10, and securing screws 11. As shown in FIG. 4, the toggle assembly 4 can include the toggle mount 10 and earplug 17.

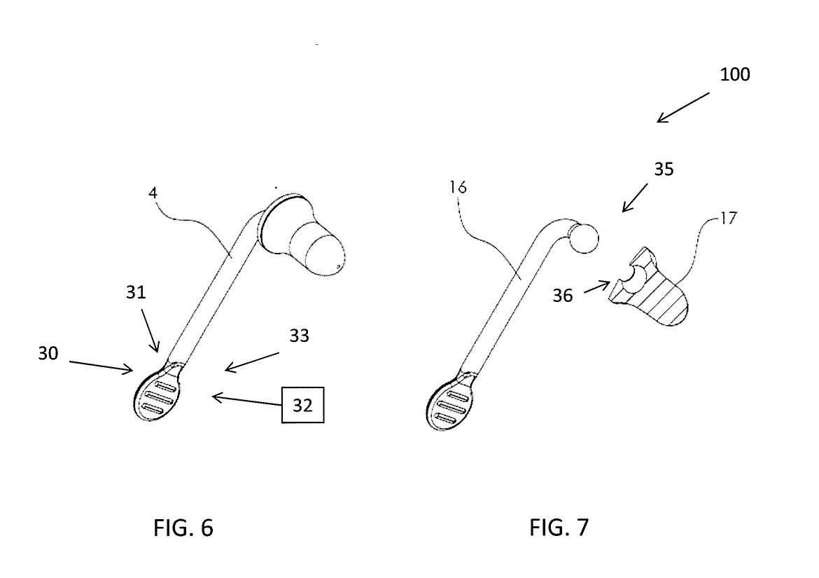

In accordance with an exemplary embodiment, the toggle mount 10 is configured to receive the toggle structure 4, which includes a toggle bar 16 with a mounting ball 35 on a distal end of the toggle bar 16 (FIGS. 6 and 7). The toggle mount 10 is received on a lower portion 41 of the earcup assembly 5. In accordance with an exemplary embodiment, the toggle structure 4 is located forward of the wearer's ear on the earmuff assemblies 1, 2 and faces backward toward the ear canal. However, in an alternative embodiment, the toggle structure 4 can face backwards and be arranged behind the wearer's ear on the earmuff 1, 2, such that the wearer accesses the toggle structure 4 from behind the ear. In accordance with an exemplary embodiment, the toggle mount 10 is preferably made of a pliable rubber material, such as silicone or high-density polyurethane foam.

In accordance with an exemplary embodiment, the left earmuff assembly 1 can be assembled by inserting the left hand ear cup assembly 5, the left hand attenuation foam mount 6, the left hand attenuation foam 7, the earmuff cushion mount 8, the earmuff cushion 9 into the left hand ear cup assembly 5 and securing them with the securing screws 11.

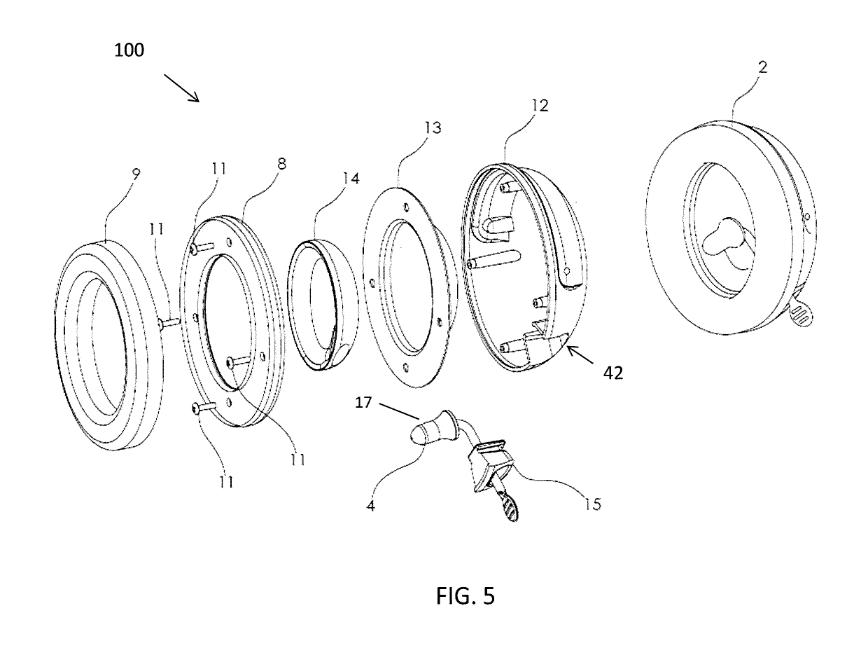

FIG. 5 is an enlarged fragmentary sectional view of the right ear muff and toggle mount with adjustable integral earplugs of FIGS. 2 and 3. As shown in FIG. 5, the right earmuff assembly 2 can include a right hand ear cup assembly 12, a right hand attenuation foam mount 13, a right hand attenuation foam 14, an earmuff cushion mount 8, an earmuff cushion 9, a right hand toggle mount 15, and securing screws 11. As shown in FIG. 5, the toggle assembly 4 can include the toggle mount 15 and the earplug 17.

In accordance with an exemplary embodiment, the right earmuff assembly 2 can be assembled by inserting the right hand ear cup assembly 12, the right hand attenuation foam mount 13, the right hand attenuation foam 14, the earmuff cushion mount 8, the earmuff cushion 9 into the right hand ear cup assembly 12 and securing them with the securing screws 11. As shown in FIG. 5, the toggle mount 10 is configured to be received in a lower portion 42 of the ear cup assembly 12.

FIGS. 6 and 7 are enlarged fragmentary sectional views of the adjustable integral earplug toggle structure 4 of FIGS. 2 and 3. As shown in FIG. 6, the adjustable integral earplug toggle structure 4 include a toggle lever 30 have a series of raised edges 32 on at least one of a front side 31 or a back side 33. The toggle structure 4 also includes a toggle bar 16 with a mounting ball 35 on a distal end. As shown in FIGS. 6 and 7, an earplug 17 with a receiving a receiving socket 36 is configured to be received on the mounting ball 35.

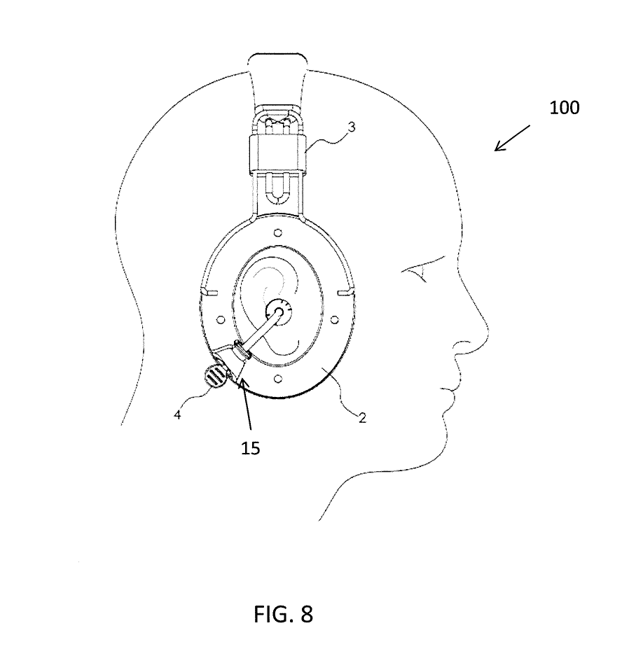

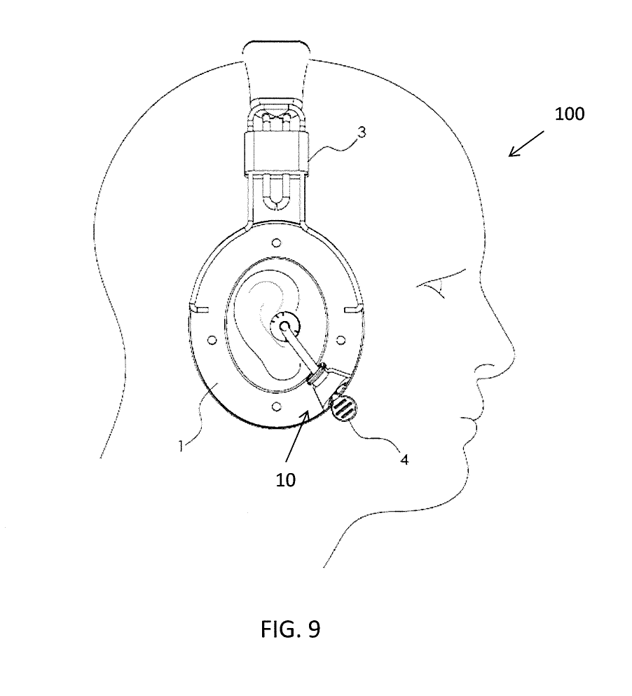

FIGS. 8 and 9 are views of the dual noise attenuation headset 100 with ear protecting muff and toggle supporting structure with adjustable integral earplugs as shown from the right, and from the right with the headset worn in the reverse position, respectively. As shown in FIGS. 8 and 9 each of the earmuff assemblies 1, 2 is preferably sized to surround and enclose an entirety of a wear's ear.

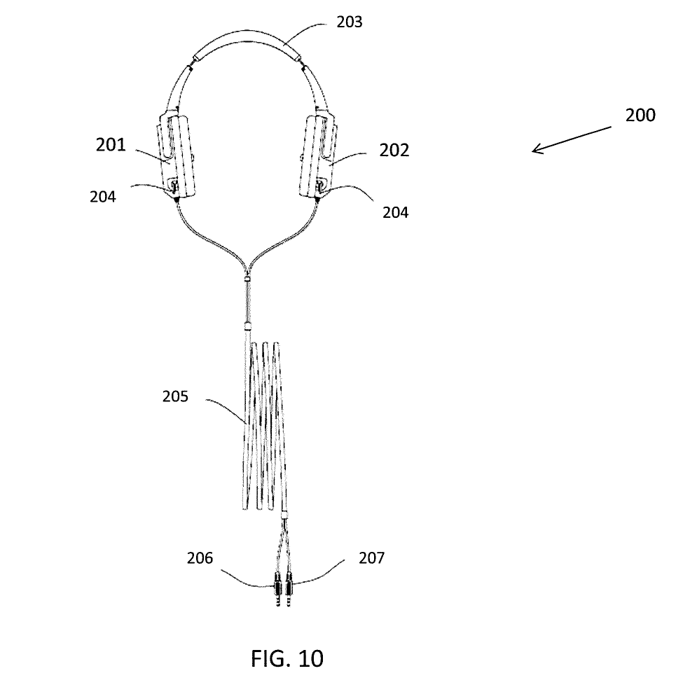

FIG. 10 is a side elevation view of a dual input quad speaker headset 200 with an ear protecting muff and toggle supporting structure with adjustable integral earplugs according to an exemplary embodiment. As shown in FIG. 10, the dual input quad speaker headset 200 can include a left headphone assembly 201, a right headphone assembly 202, a headband assembly 203, and a toggle assembly 204 for each of the left and right headphone assemblies 201, 202. As shown in FIG. 10, the headband assembly 203 attaches the right and the left earmuff assemblies 201, 202 to one another. In accordance with an exemplary embodiment, the headband assembly 203 is configured to be worn on the top of the user's head. The headset 200 can also include, for example, a two line, four input cord 205, a large speaker input plug, which carries left and right hand audio signals 206, and an earbud speaker input plug, which carries left hand and right hand audio signals 207.

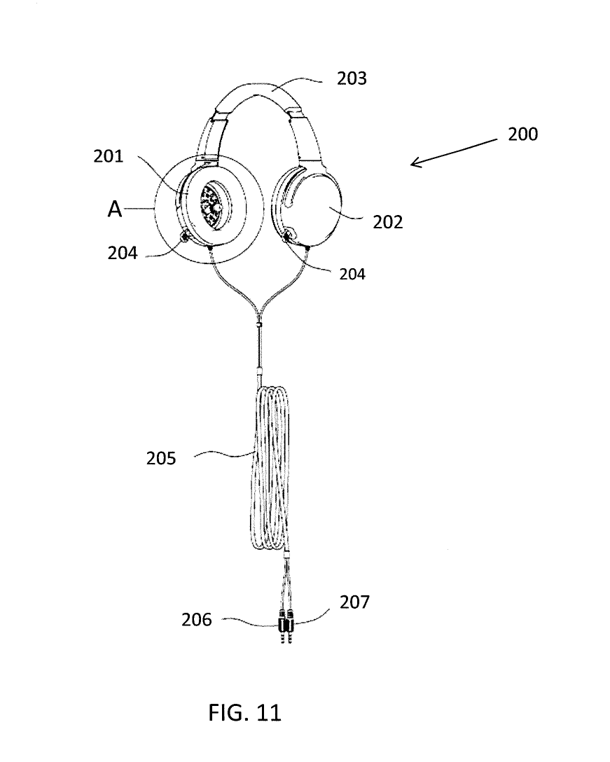

FIG. 11 is an elevation view of the dual input quad speaker headset 200 of FIG. 10 with an ear protecting muff and toggle supporting structure with adjustable integral earplugs according to an exemplary embodiment. As shown in FIG. 11, the dual input quad speaker headset 200 can include the left headphone assembly 201, the right headphone assembly 202, the headband assembly 203, and the toggle assembly 204 for each of the left and right headphone assemblies 201, 202. The headset 200 can also include the two line, four input cord 205, the large speaker input plug 206, which carries left and right hand audio signals, and the earbud speaker input plug 207, which carries left hand and right hand audio signals. The audio signals received from the input plug 206, 207, can be one or more independent audio signals or audio channels, which can include a same and/or different inputs or audio channels. In accordance with an exemplary embodiment, the audio signals from the input plugs 206, 207 can produces, for example, two or more stereo sounds for a stereophonic sound, or a stereo sound and conversational communication, for example, in a teaching environment, for example, learning to play the piano or guitar while simultaneously hearing the feedback for the instrument and the digital instruction from a different device, such as an iPad, iPhone or computer.

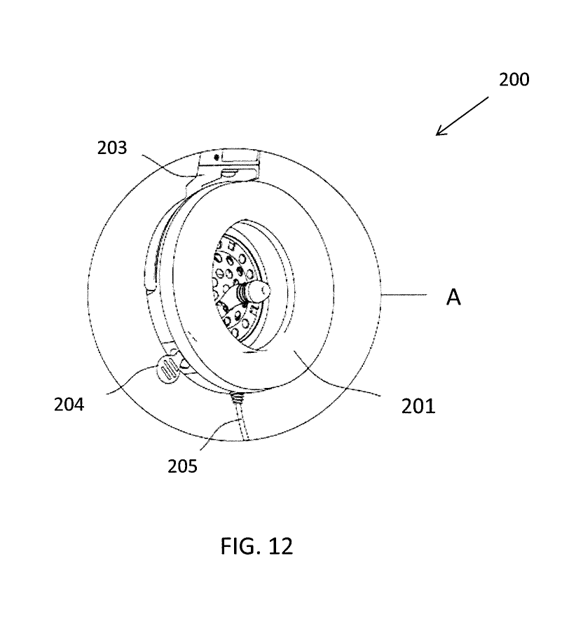

FIG. 12 is an elevation view of a left headphone assembly 201. As shown in FIG. 12, the left headphone assembly 201 can include the headband assembly 203, the toggle assembly 204, and the two line, four input cord 205.

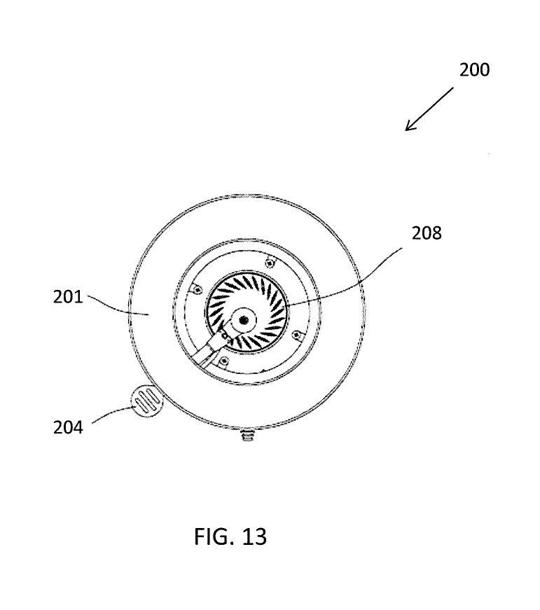

FIG. 13 is a side elevation view of the left headphone assembly 201 as shown in FIG. 12. As shown in FIG. 13, the left headphone assembly 201 can include the toggle assembly 204 and a large speaker 208, which is arranged inside of the left headphone assembly 201.

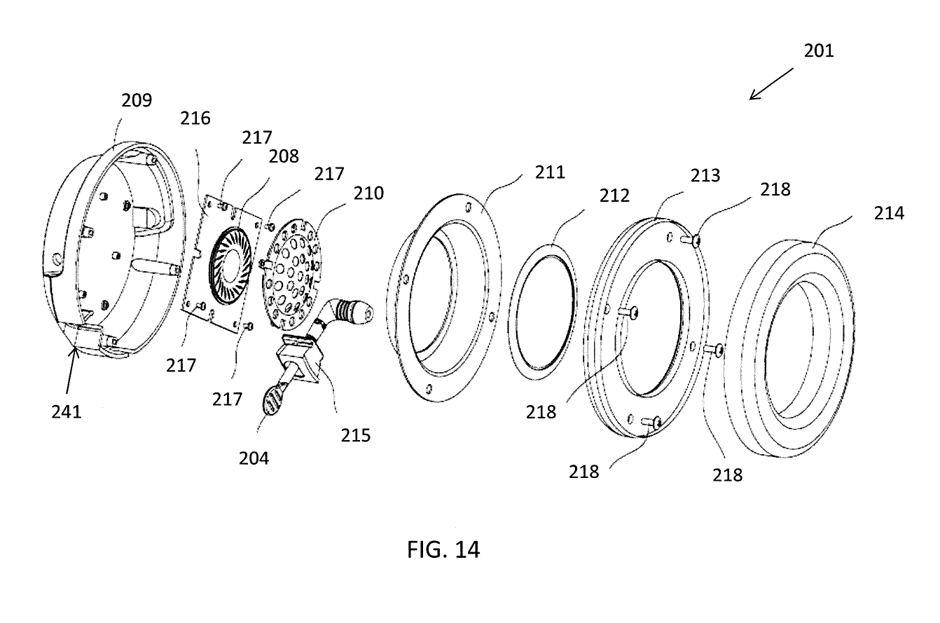

FIG. 14 is an enlarged fragmentary sectional view of the left headphone assembly 201. As shown in FIG. 14, the left headphone assembly 201 can include a left hand earcup assembly 206, a left hand speaker grill 210, a left hand attenuation foam mount 211, a left hand attenuation foam 212, a headphone cushion mount 213, a headphone cushion 214, a left hand toggle mount 215, a left hand speaker mount (or printed circuit board) 216, a plurality of speaker (PCB) securing screws 217, and a plurality of headphone cushion securing screws 218.

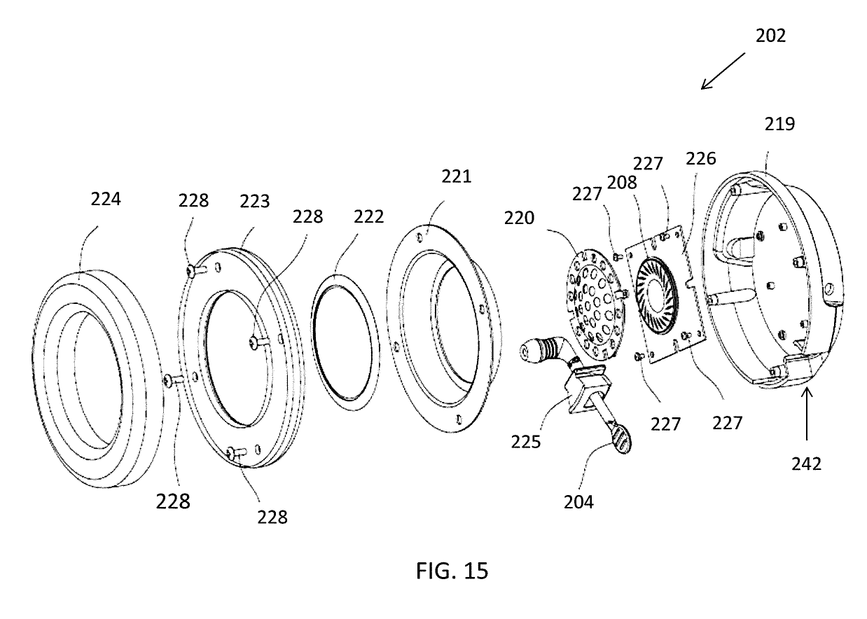

FIG. 15 is an enlarged fragmentary sectional view of the right headphone assembly 202. As shown in FIG. 15, the right headphone assembly 202 can include a right hand earcup assembly 219, a right hand speaker grill 220, a right hand attenuation foam mount 221, a right hand attenuation foam 222, a headphone cushion mount 223, a headphone cushion 224, a right hand toggle mount 225, a right hand speaker mount (or printed circuit board) 226, a plurality of speaker (PCB) securing screws 227, and a plurality of headphone cushion securing screws 228.

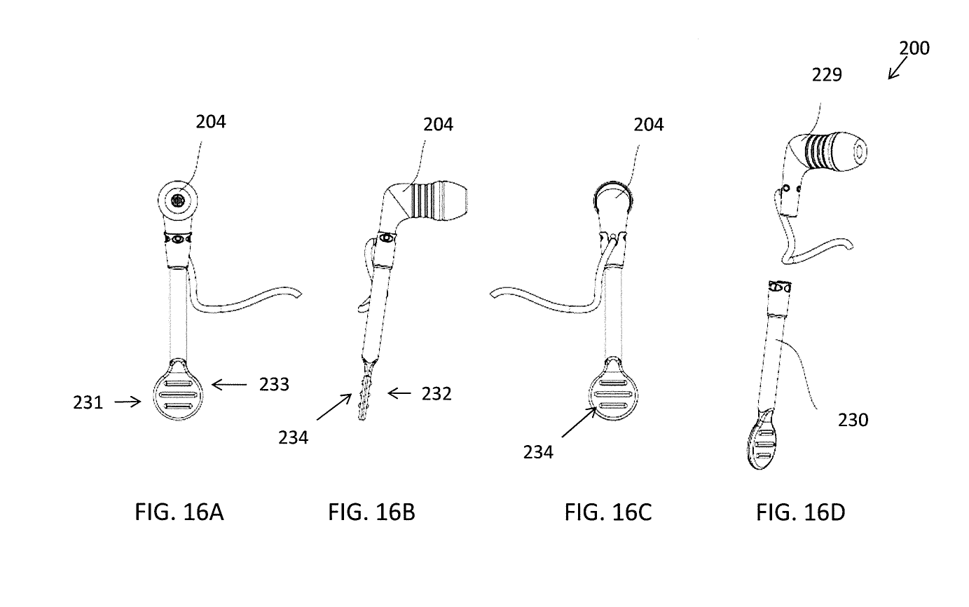

In accordance with an exemplary embodiment, the toggle mount 215, 225 is configured to receive the toggle structure 204, which includes a toggle bar 16 with a mounting ball 35 on a distal end of the toggle bar 230 (FIGS. 16A-16D). The toggle mount 215, 225 is received on a lower portion 241, 242 of the earcup assembly 209, 219. In accordance with an exemplary embodiment, the toggle structure 214 is located forward of the wearer's ear in the earmuff 201, 202 and faces backwards toward the ear canal. However, in an alternative embodiment, the toggle structure 214 can face backwards and be arranged behind the wearer's ear on the earmuff 201,202, such that a wearer accesses the toggle structure 214 from behind the ear. In accordance with an exemplary embodiment, the toggle mount 215, 225 is preferably made of a foam or rubber material, such as a pliable rubber material, such as silicone or high-density polyurethane foam.

In accordance with an exemplary embodiment, each of the large speakers (or speakers) 208 within the right and left headphone assembles 201, 202 is configured to receive an audio signal from one of the two line, four input cords 205, or alternatively configured to receive an audio signal via Bluetooth® or other known wireless technology.

FIGS. 16A-16D are enlarged fragmentary sectional views of the adjustable integral earplug toggle structure 4 of FIGS. 11 and 12. As shown in FIGS. 16A-16D, the adjustable integral earplug toggle structure 4 includes a toggle lever 231 have a series of raised edges 233 on at least one of a front side 232 or a back side 234. The toggle structure 4 also includes a toggle bar 230 with an earbud speaker 229 on a distal end of the toggle bar 230. In accordance with an exemplary embodiment, each of the earbud speakers 229 is configured to receive an audio signal from one of the two line, four input cords 205, or alternatively configured to receive an audio signal via Bluetooth® or other known wireless technology.

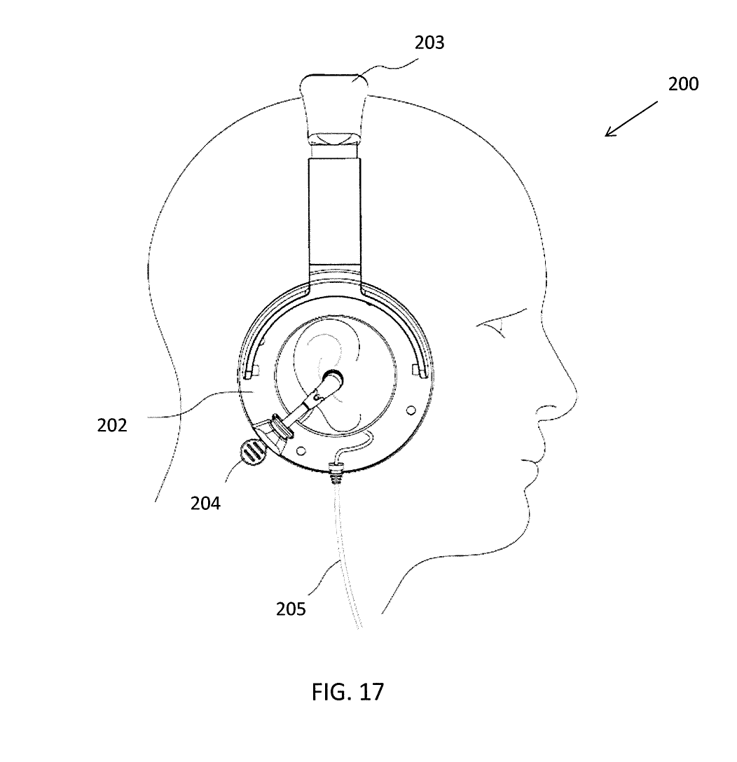

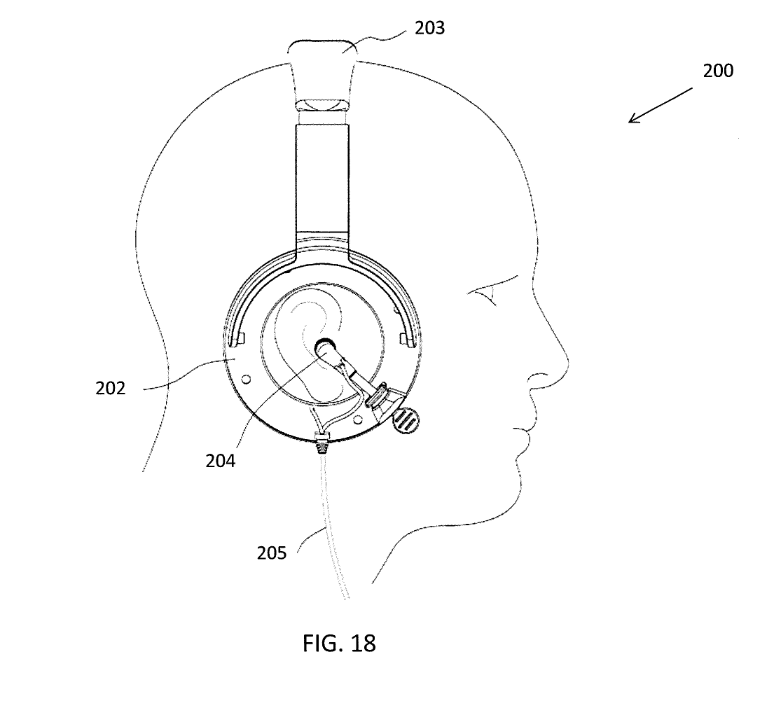

FIGS. 17 and 18 are views of the dual input quad speaker headset 200 with ear protecting muff and toggle supporting structure with adjustable integral earplugs as shown from the right, and from the right with the headset worn in the reverse position. As shown in FIGS. 17 and 18, each of the earmuff assemblies 202, 203 is preferably sized to surround and enclose an entirety of a wear's ear.

In accordance with an exemplary embodiment, the dual noise attenuation headset 100 and the dual input quad speaker headset 200 are primarily made of a plastic material and can be molded or formed using 3D printing technology with the exception, for example, of the toggle mounts 10, 20, 215, 225, and noise attenuation components and/or foam materials 9, 214, 224, which are foams or rubber materials as disclosed herein.

In accordance with an exemplary embodiment, the dual noise attenuation (DNA) headset 100 and the dual input quad speaker (DIQS) headset 200 can include materials or designs of different frequency attenuation potential that, when combined, offer a broader spectrum of noise dampening. For example, one part of the dual noise attenuation headset 100 could be optimized for the sound, for example, below 500 Hz, and the other for reductions, for example, between 500 Hz and 2000 Hz. In accordance with an exemplary embodiment, the headset 100, 200 can be extended to work together above 2000 Hz, for example, in the bone conduction range. Since the headsets 100, 200 are designed to always be worn together (unlike mixing of different non-complimentary earmuff and earplugs), the important mechanical interaction effects can be reduced, improving the overall noise reduction (NRR). In addition, having dual noise protection in a single product eliminates the annoyance of trying to comply with HPD mandates for exposure above 100 Dba that requires dual protection from mismatched products and cumbersome installation. In addition, the dual input quad speaker (DIQS) 200 can extend the frequency listening range of a typical headset, for example, by having the earbuds optimized for high frequency inputs (i.e., a tweeter speaker), and the headphones optimized for low frequency inputs (i.e., a base speaker).

In accordance with an exemplary embodiment, components of the headset 100, 200, for example, the earmuff assemblies 1, 2, 202, 203 can be part of a kit that can retrofit, for example, non DNA headsets and/or earmuffs. In accordance with an exemplary embodiment, the earmuff assemblies 1, 2, 202, 203 can also be fitted with active electronic noise reduction components, as well as passive methods.

It will be apparent to those skilled in the art that various modifications and variation can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A dual attenuation ear protecting headgear, the headgear comprising:
a pair of earmuff assemblies;
a headband assembly for connecting the pair of earmuff assemblies to one another; and
means for externally adjusting a location of an integral inner earplug into the ear canal of a wearer in each of the pair of earmuff assemblies.

2. The headgear of claim 1, wherein the means for adjusting the location of the integral inner earplug is a toggle assembly, the toggle assembly comprising a toggle bar with an earplug on a distal end.

3. The headgear of claim 2, wherein the toggle assembly is received through a toggle mount having a cavity configured to receive the toggle bar.

4. The headgear of claim 2, comprising:
a toggle lever on each proximal end of the toggle bar, and wherein the toggle lever is configured to be outside of the earmuff.

5. The headgear of claim 4, wherein a user can be manually manipulate the toggle assembly, which selectively manipulates an end of the earplug into the wearer's ear.

6. The headgear of claim 2, wherein the earplugs are replaceable.

7. A dual attenuation ear protecting headgear, the headgear comprising:
a pair of earmuff assemblies;
a headband assembly for connecting the pair of earmuff assemblies to one another; and
a toggle assembly for externally adjusting a location of an integral inner earplug into the ear canal of a wearer in each of the pair of earmuff assemblies.

8. The headgear of claim 7, wherein toggle assembly comprises a toggle bar with an earplug on a distal end.

9. The headgear of claim 8, wherein the toggle assembly is received through a toggle mount having a cavity configured to receive the toggle bar.

10. The headgear of claim 8, comprising:
a toggle lever on each proximal end of the toggle bar, and wherein the toggle lever is configured to be outside of the earmuff.

11. The headgear of claim 10, wherein a user can be manually manipulate the toggle assembly, which selectively manipulates an end of the earplug into the wearer's ear.

12. The headgear of claim 8, wherein the earplugs are replaceable.

13. A dual input quad headphone assembly, the headphone assembly comprising:
a pair of earmuff assemblies, each of the pair of earmuff assemblies having a speaker configured to receive an audio signal;
a headband assembly for connecting the pair of earmuff assemblies to one another; and
means for externally adjusting a location of an integral inner earbud into the ear canal of a wearer in each of the pair of earmuff assemblies, each earbud receiving an audio signal.

14. The headphone assembly of claim 13, wherein the means for adjusting the location of the integral inner earplug is a toggle assembly, the toggle assembly comprising a toggle bar with an earbud on a distal end.

15. The headphone assembly of claim 14, wherein the toggle assembly is received through a toggle mount having a cavity configured to receive the toggle bar.

16. The headphone assembly of claim 14, comprising:
a toggle lever on each proximal end of the toggle bar, and wherein the toggle lever is configured to be outside of the earmuff.

17. The headphone assembly of claim 16, wherein a user can be manually manipulate the toggle assembly, which selectively manipulates an end of the earbud into the wearer's ear.
